(19) United States
(12) Patent Application Publication
(10) Pub. No.: US 2024/0091122 A1
(43) Pub. Date: Mar. 21, 2024

(54) Growth Factor Formulation

(71) Applicant: Topix Pharmaceuticals, Inc., North Amityville, NY (US)

(72) Inventor: Steven M. Hernandez, Blue Point, NY (US)

(21) Appl. No.: 18/368,937

(22) Filed: Sep. 15, 2023

Related U.S. Application Data
(60) Provisional application No. 63/407,220, filed on Sep. 16, 2022.

(51) Int. Cl. A61K 8/64; A61K 8/35; A61K 8/97; A61Q 19/00

(52) U.S. Cl. CPC A61K 8/645; A61K 8/355; A61K 8/97; A61Q 19/00

(57) ABSTRACT

Disclosed in certain embodiments herein is a formulation including a plant-based growth factor and at least one of a peptide, an antioxidant and a botanical agent. The formulation has been found to improve the properties of the skin on the face and on areas under the eye. Also disclosed herein are methods of administering the formulation described herein for treating a skin.

### CROSS REFERENCE TO RELATED APPLICATION(S)

The present application claims priority to U.S. Provisional Patent Application No. 63/407,220 filed on Sep. 16, 2022, the entire contents of which are incorporated herein in its entirety.

### FIELD OF THE INVENTION

The present invention relates to cosmetic compositions for skin care applications. In particular, the cosmetic compositions include a plant-based growth factor for use in skin care applications.

### BACKGROUND OF THE INVENTION

A variety of cosmetic topical compositions have been developed over the years to treat skin on the face or in under the eye areas. Growth factors are a common ingredient in some formulations as they are natural substances that support the repair of damaged skin. Growth factors also promote the formation of collagen and elastin to provide firmness and elasticity Some attempts have been used to develop topical compositions using human growth factors. However, human growth factors have been found to cause negative side effects, such as growing unwanted facial hair or a tumor. Human growth factors include large molecules that may be too large to penetrate intact skin. Additionally, when human growth factors are continually used, skin health related concerns arise. Human growth factors are also known for having an unpleasant scent and user experience. Further, human-derived growth factors may be derived from controversial sources.

Thus, there is a need to develop a formulation that can utilize the benefits of growth factors, while limiting the risks.

### SUMMARY

It is an object of certain embodiments of the disclosure to provide a topical composition, and a method of using said topical composition for protecting the skin. It has been found that using plant-based growth factors unexpectedly provided substantial improvements to the skin when used in combination with at least one of a peptide, an antioxidant or a botanical agent.

It is an object of certain embodiments of the disclosure to provide a formulation for improving the appearance of the skin.

In an embodiment of the present disclosure, a formulation is provided. The topical composition may be a formulation including at least one plant-based growth factor and at least one of a peptide, an antioxidant or a botanical agent.

In some embodiments of the formulation, the plant-based growth factor may include a plant-based synthetic human epidermal growth factor (EGF), a plant-based synthetic vascular endothelial growth factor (VEGF), a plant-based synthetic transforming growth factor (TGF), a plant-based synthetic insulin growth factor (IGF), or a combination thereof.

In some embodiments of the formulation the peptide may include a hexapeptide, a tripeptide, a pentapeptide, or a combination thereof. The hexapeptide may be acetyl hexapeptide-8, or hexapeptide-40. The hexapeptide-40 may be Nicotiana benthamiana Hexapeptide-40 sh-Oligopeptide-1 or Nicotiana benthamiana Hexapeptide-40 sb-Polypeptide-76.

In some embodiments, the tripeptide may be palmitoyl tripeptide-5. The pentapeptide may be palmitoyl pentapeptide-4.

In some embodiments of the formulation, the peptide may be acetyl hexapeptide-8, a hexapeptide-40, palmitoyl tripeptide-5, palmitoyl pentapeptide-4, or a combination thereof. In another embodiment of the formulation, the peptide may be acetyl hexapeptide-8, a hexapeptide-40, or a combination thereof.

In some embodiments of the formulation, the antioxidant may be ubiquinone.

In some embodiments of the formulation, the botanical agent may include at least one of a chamomile extract, a cucumber extract, a green tea extract, a seaweed extract, or a combination thereof.

In some embodiments, the formulation may further include an alcohol. The alcohol may include butylene glycol, ethyl alcohol, or isopropyl alcohol.

In some embodiments, the formulation may further include a humectant. The humectant may be glycerin.

In some embodiments, the formulation may further include purified water. In some embodiments, the formulation may further include a chelating agent. The chelating agent may be disodium ethylenediaminetetraacetic acid (EDTA).

In some embodiments, the formulation may further include a pH stabilizer. The pH stabilizer may be potassium hydroxide, sodium hydroxide, or a combination thereof.

In some embodiments, the formulation may further include a polymer. The polymer may be a crosspolymer. The crosspolymer may be a poly(methlvinylether/maleic anhydric decadiene) crosspolymer.

In some embodiments, the formulation may further include a preservative. The preservative may be phenoxyethanol.

In some embodiments, the formulation may further include a surfactant. The surfactant may be a non-ionic surfactant, or an ionic surfactant. The non-ionic surfactant may include polysorbate 20.

In some embodiments, the formulation may further include sodium hyaluronate.

In some embodiments, the formulation may be a cream, gel, emulsion, serum, or lotion.

In some embodiments of the formulation, the peptide may be included in an amount of about 10 wt % to about 50 wt %, about 15 wt % to about 45 wt %, about 20 wt % to about 40 wt %, or about 25 wt % to about 35 wt %, based on total weight of the formulation.

In some embodiments of the formulation, the plant-based growth factor may be included in an amount of 1 wt % to about 10 wt %, about 2 wt % to about 9 wt %, about 3 wt % to about 8 wt %, or about 4 wt % to about 6 wt %, based on total weight of the formulation.

In some embodiments of the formulation, the antioxidant may be included in an amount of about 0.0001 wt % to about 1 wt %, about 0.001 wt % to about 0.9 wt %, about 0.05 w % to about 0.5 wt %, or about 0.01 wt % to about 0.05 wt %, based on total weight of the formulation.

In some embodiments of the formulation, the botanical agent may be included in an amount of about 0.0001 wt % to about 1 wt %, about 0.001 wt % to about 0.5 wt %, about 0.01 wt % to about 0.1 wt %, or about 0.02 wt % to about 0.07 wt %, based on total weight of the formulation.

In another embodiment a method of treatment is provided. The method of treatment may include applying a formulation include a plant-based growth factor to the skin.

In another embodiment, a method of treatment is provided including applying a formulation including a plant-based growth factor to the face.

In another embodiment, a method of treatment is provided including applying a formulation including a plant-based growth factor to an area under the eye.

In another embodiment, a method of treatment is provided including applying a first formulation including a plant-based growth factor to the skin and applying a second formulation including a plant-based growth factor to the skin. The first formulation may include a peptide mixture including acetyl hexapeptide-8, a hexapeptide-40, and a tripeptide, wherein the tripeptide is palmitoyl tripeptide-5. The second formulation may include a peptide mixture acetyl hexapeptie-8, a hexapeptide-40, and a tripeptide and at least one plant-based growth factor, wherein the tripeptide is palmitoyl tripeptide-5, palmitoyl pentapeptie-4, or a combination thereof

The term “administering the formulation” as used herein refers to applying topically onto a skin of a subject, e.g., on the face, under the eyes, neck, hands, feet, elbows, knees, and the like. As used herein, the terms “application,” “apply,” and “applying” with respect to a disclosed topical formulation or method of using a disclosed topical formulation, refer to any manner of administering a topical formulation to the skin, for example, the skin of a person, such as the skin of a patient, which, in medical or cosmetology practice, delivers the formulation to the subject's skin surface. Smearing, rubbing, spreading, spraying a disclosed topical formulation, with or without the aid of suitable devices, on a subject's skin are all included within the scope of the term “application,” as used herein. The term “topical” or “topically” with respect to administration or application of a disclosed skincare formulation refers to epicutaneous administration or application, onto skin. The application can be manually (e.g., directly with the hands) or manipulated with an applicator, cloth, device, roll-on, wipes, unit dose sponge applicators, liquid applied with swabs or cotton balls, impregnated gauze or other substrates, coated silicone sheets or other sheet goods, coated bandages or externally fixed devices, towelettes, individually packages pledgettes or pads, transdermal delivery system, etc. Administration can be self-administration or administration by a medical professional or caregiver.

### DETAILED DESCRIPTION

All percentages, parts and ratios used herein are by weight of the total composition and all measurements made are at room temperature, unless otherwise specified. All percentages, parts, and ratios are based upon the total weight of the compositions disclosed herein, unless otherwise specified.

As used herein, the terms “treatment” or “treating” with respect to a skin condition generally mean “having positive effect on a skin condition” and encompass reduction, amelioration, and/or alleviation of at least one symptom of a skin condition, a reduction, amelioration, and/or alleviation in the severity of the skin conditions, or delay, prevention, or inhibition of the progression of the skin condition. Treatment, as used herein, therefore does not require total curing of the condition. A formulation of the present disclosure that is useful for treatment of a skin condition, or a method of treating a skin condition, need only reduce the severity of a skin condition, reduce the severity of symptoms associated therewith, provide improvement to a patient's quality of life, or delay, prevent, or inhibit the onset of one or more symptoms of a skin condition. As used herein, these terms also encompass aesthetic improvements to the skin upon application of the disclosed formulations having a plant-based growth factor.

As used herein, the terms “application,” “apply,” and “applying” with respect to a disclosed topical formulation or method of using a disclosed topical formulation, refer to any manner of administering a topical formulation to the skin, for example, the skin of a person, such as the skin of a patient, which, in medical or cosmetology practice, delivers the formulation to the subject's skin surface. Smearing, rubbing, spreading, spraying a disclosed topical formulation, with or without the aid of suitable devices, on a subject's skin are all included within the scope of the term “application,” as used herein. The term “topical” or “topically” with respect to administration or application of a disclosed skincare formulation refers to epicutaneous administration or application, onto skin.

As used herein, the phrase “effective amount” refers to an amount of a formulation of the present disclosure, or component thereof, effective to treat a skin condition as noted above, including a range of effects, from a detectable local improvement in an area of topical application to substantial relief of symptoms to an improvement in one or more aesthetic criteria, including, but not limited to, a perceived improvement in apparent skin dryness, keratosis pilaris, age, radiation damage, sun or uv damage, skin tone, weather-beaten appearance, yellowing, loss of elasticity, redness, dryness, age spots, skin wrinkles, skin smoothness, brightness, radiance, as well as skin pores becoming less noticeable. The effective amount will vary with the particular condition or conditions being treated, the severity of the condition, the duration of the treatment, the specific components of the composition being used, and other factors. More specifically, the disclosed compositions and formulations provide a method for therapeutic treatment of skin by providing, in some embodiments, plant-based growth factors in an efficacious manner to the skin. The disclosed compositions, formulations and methods of use thereof reduce, minimize, or eliminate normally-observed dry skin conditions including, inter alia, conditions characterized by keratosis pilaris, itching, severe skin flaking, breakdown of the skin barrier, discomfort, extreme dryness, cracking of the skin and sensitization. The disclosed compositions, formulations, and methods of use thereof also provide aesthetic improvements in the skin, including but not limited to skin that appears younger, skin exhibiting a more even tone, skin in which the pores are less noticeable, and skin that is judged by the user to be smoother, and/or to be improved with respect to its weather-beaten or aged appearance, yellowing, loss of elasticity, redness, dryness, age spots, and/or skin wrinkles.

As used herein, the term “plant-based synthetic” when referencing growth factors refers to a botanical plant that is bioengineering such that the botanical plant, or extract therefrom, functions as a growth factor

A natural process of aging causes a decline in epidermal thickness, resulting in unwanted skin laxity with visual signs including lines, wrinkles and/or sagging. This is because as people get older, the cell renewal rate slows down meaning that new cells take longer to reach the surface, i.e. surface of the skin. Additionally, outside factors can also effect the skin including being exposed to cold temperatures, humidity, wind, heat and sun. These factors can lead to fine lines and/or wrinkles. A person may also experience dull, blotchy, freckled and/or uneven skin tone. Additionally, severe pigmentation concerns including hyperpigmentation and post-inflammatory hyperpigmentation may occur. Dead skin cells may also buildup on the outer surface of the skin, which may cause rough skin texture, lines, wrinkles and/or enlarged pore size. As people age, skin discoloration and sensitivity may occur as well, including, but not limited to, eczema, psoriasis, rosacea, or acute trauma from excess sun exposure.

Growth factors are naturally present in the skin. Growth factors prompt intercellular communication in the skin, which aids in maintaining healthy skin cells and promoting collagen production. Growth factors are proteins that may be derived by plants or humans. When added to topical compositions, growth factors may regulate cellular growth, proliferation and/or differentiation. Growth factors may be present in all cell types in both the epidermis and dermis of the skin including keratinocytes, fibroblasts, and melanocytes. By including growth factors in a topical composition, several benefits may occur. For example, improving overall skin rejuvenation, reducing the appearance of fine lines and wrinkles, improving the appearance of skin tone and texture, optimizing collagen production, and/or improving the appearance of skin firmness and elasticity.

Plant-based growth factors have been found to provide an accelerated advantage over human derived growth factors by adding an increased level of precision. This results in an efficacious, balanced blend of skin restoring proteins. As will be described herein, the formulation of the present disclosure includes at least one plant-based growth factor, which has several benefits. The benefits include, but are not limited to, visibly reduces fine lines and wrinkles; skin appears firmer instantly, restoring elasticity; supports the skin barrier and new collagen production; hydrates and protects against free radical damage; supports rate of recovery post procedure; and visibly reduces redness and erythema.

According to an embodiment, a formulation including at least one plant-based growth factor of the present disclosure has a clear advantage over a formulation including human-derived growth factors. That is, a plant-based growth factor provides the ability to control the concentration and increase level of precision, with the ability to target specific skin pathways. Therefore, when a formulation includes a plant-based growth factor, it was found to result in an efficacious, balanced blend of skin-restoring protection with predictable, visible results.

According to various embodiments, the present disclosure relates to a formulation that includes at least one plant-based growth factor and at least one of a peptide, an antioxidant, or a botanical agent.

In some embodiments of the formulation, the at least one plant-based growth factor may include plant-based synthetic human epidermal growth factor (EGF), a plant-based synthetic vascular endothelial growth factor (VEGF), a plant-based synthetic transforming growth factor (TGF), a plant-based synthetic insulin growth factor (IGF), or a combination thereof.

Including at least one plant-based growth factor in a topical formulation was found to provide controlled, precision upregulation of skin improvement benefits; faster visible results with continued usage; promote skin rejuvenation by restoring balance to the collagen ratio; unlocks skin's ability to naturally recover and restore balance; and/or has a pleasant scent.

In some embodiments of the formulation, the peptide may be a hexapeptide, a tripeptide, a pentapeptide, or a combination thereof. In some embodiments, the hexapeptide may be acetyl hexapeptide-8 and a hexapeptide-40. The hexapeptide-40 may be Nicotiana benthamiana Hexapaeptide-40 sh-Oligopeptide-1 or Nicotiana benthamiana Hexapeptide-40 sh-Polypeptide-76. The tripeptide may be palmitoyl tripeptide-5. The pentapeptide may be palmitoyl pentapeptide-4.

In certain embodiments, the peptide may be acetyl hexapeptide-8, a hexapeptide-40, palmitoyl tripeptide-5, palmitoyl pentapeptide-4, or a combination thereof. In other embodiments, the peptide may be acetyl hexapeptide-8, a hexapeptide-40, or a combination thereof.

In some embodiments of the formulation, the antioxidant may be polyphenols, ascorbates, tetrahexyldecyl ascorbate, ubiquinone, green tea phenols, cinnamic acid, ferulic acid, caffeic acid, p-coumaric acid, sinapinic acid, cis and trans isomers thereof, salts thereof, equivalent derivatives thereof, and combinations thereof. In some embodiments, the antioxidant may be gallic acid, delphinidin, luteolin, quercetin, cyanidin, taxifolin, kaempferol, malvidin, hesperidin, pelargonidin, apigenin, naringenin, chrysin, ergothioneine, glutathione, emblica, cis and trans isomers thereof, salts thereof, equivalent derivatives thereof, and combinations thereof. In certain embodiments, the antioxidant may be apigenin, ergothioneine, glutathione, emblica, cis and trans isomers thereof, salts thereof, equivalent derivatives thereof, and combinations thereof. In some embodiments, the antioxidant may be anserine, ascorbic acid, butylated hydroxyanisole (BHA), butylated hydroxytoluene (BHT), cysteine, diosmine, hydroquinone, kojic acid, palmatine, pectolinarin, tannic acid, ectoine, ubiquinone, cis and trans isomers thereof, salts thereof, equivalent derivatives thereof, and combinations thereof.

In some embodiments of the formulation, the botanical agent may be at least one of a chamomile extract, a cucumber extract, a green tea extract, a seaweed extract, or a combination thereof. In other embodiments of the formulation, the botanical agent may be an aloe extract, an oat extract, a beet extract, an orange extract, a grapefruit extract, a lemon extract, a coconut extract, or a dandelion extract.

In some embodiments, the formulation may further include a humectant. Humectants are hygroscopic substances used to keep things moist, often a molecule with several hydrophilic groups, or capable of hydrogen bonding with water, or having other polar organic functional groups. Humectants can also function as solvents or cosolvents.

Some examples of humectants include, but are not limited to, amino acids; glycerin; glycols and polyols, such as propylene glycol, hexylene glycol, and butylene glycol, including polymeric and sugar-based polyols/alcohols, for example, glycerol, sorbitol, xylitol, maltitol, polydextrose; mucopolysaccharides and carbohydrates, for example, aloe vera gel, yucca extract, dextrose and polydextrose; alpha hydroxy acids such as glycolic acid, lactic acid; albumen; esters or amides of acetic acid or similar; soluble carbohydrates, such as sugar, honey; salts, such as potassium chloride, sodium PCA, salts of polycarboxylic acids; amides, such as urea and urea derivatives.

In some embodiments, the formulation may include sodium hyaluronate, such as fractionated and long-chain sodium hyaluronate, from single monomer to about 5 million Daltons.

In certain embodiments, the formulation further includes a cosmetically acceptable excipient. Exemplary cosmetically acceptable excipients, include, without limitations, pH stabilizer, epidermal penetration enhancer, solvent, mild surfactants, oil bodies, emulsifiers, pearlescent waxes, consistency regulators, thickeners, rheology modifiers, suspending agents, chelating agents, preservatives, super fatting agents, stabilizers, polymers, silicone or siloxane compounds, fats, waxes, lecithins, phospholipids, Saccharomyces lysate extract, UV photoprotective factors, biogenic active ingredients, additional antioxidants, film formers, swelling agents, insect repellents, tyrosinase inhibitors, hydrotropes, solubilizers, perfume oils, dyes, zinc oxide, fatty alcohols, esters of fatty acids, adjuvants, Natural or Synthetic Triglycerides Including Glyceryl Esters and Derivatives, hydrocarbon oils, super-fatting agents, polymers, biogenic active ingredients, hydrotropic agents, bacteria-inhibiting agents, colorants, UV screening agents, agents that absorb UV light and provide photo protection to the skin, or combinations thereof. In certain embodiments, the formulation includes a cosmetically acceptable excipient selected from the group of solvents, emulsifiers, consistency regulators, thickeners, suspending agents, additional antioxidants, preservatives, perfume oils, or a combination thereof.

In some embodiments, the pH stabilizer may be potassium hydroxide, sodium hydroxide, or a combination thereof.

Suitable solvents that may be used in the formulation described herein include, without limitations, water, alkanediols (e.g., ethylene glycol, propylene glycol, butylene glycol), ethoxylated or propoxylated diglycol, ethanol, propanol, isopropanol, glycerin, methoxyisopropanol, PPG-2 methyl ether, PPG-3 methyl ether, propylene glycol butyl ether, PPG-2 butyl ether, phenoxyisopropanol, butoxyethanol, butoxydiglycol, methoxydiglycol, phenoxyethanol, PPG-3 butyl ether, PPG-2 propyl ether, propylene glycol propyl ether, or dipropylene glycol dimethyl ether, or mixtures and combinations or individual ethoxylates, propoxylates and glyceryl esters thereof. In some embodiments, the solvent may be butylene glycol, ethyl alcohol, isopropyl alcohol, or a combination thereof.

In certain embodiments, the solvent includes, comprises, consists, or consists essentially of an alkane diol (such as ethylene glycol, propylene glycol, butylene glycol), glycerin, water, or a mixture thereof. In one embodiment, the solvent includes, comprises, consists, or consists essentially of glycerin, propylene glycol, isopentyl diol, and water.

In certain embodiments, the cosmetically acceptable excipient includes natural gums (e.g., a natural plant gum). Suitable natural gums include, without limitations, guar gum, carob gum, konjac gum, xanthan gum, Sclerotium gum, acacia gum, cellulose gum (modified or not), or a combination thereof. In one embodiment, the natural gum in the topical composition is Sclerotium gum. The natural gum contributes to the texture of the topical composition and may also act as a suspending agent for the oily phase in the aqueous phase of the emulsion.

In certain embodiments, the cosmetically acceptable excipient includes an emulsifier. Suitable emulsifiers include, without limitations, polysorbate 20, cetearyl alcohol, polysorbate 60, stearyl alcohol, glyceryl stearate, sodium polyacrylate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, Glyceryl Stearate Citrate, Glyceryl Stearate SE (self-emulsifying), stearic acid, salts of stearic acid, polyglyceryl-3-methylglycosedistearate, or a combination thereof.

Further suitable emulsifiers are phosphate esters and the salts thereof such as cetyl phosphate (Amphisol® A), diethanolamine cetyl phosphate (Amphisol® DEA), potassium cetyl phosphate (Amphisol® K), sodium cetearyl sulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Cetearyl Glucoside, Lauryl Glucoside, Decyl Glucoside, Sodium Stearoyl Glutamate, Sucrose Polystearate and Hydrated Polyisobutene. Furthermore, one or more synthetic polymers may be used as an emulsifier. For example, PVP eicosene copolymer, acrylates/C10-30 alkyl acrylate crosspolymer, acrylates/steareth-20 methacrylate copolymer, PEG-22/dodecyl glycol copolymer, PEG-45/dodecyl glycol copolymer, and mixtures thereof.

In certain embodiments, the cosmetically acceptable excipient includes a chelating agent. Suitable chelating agents include, without limitations, disodium ethylenediaminetetraacetic acid (EDTA), diethylenetriaminepentaacetic acid (DTPA), N-(hydroxyethyl)-ethylenediaminetriacetic acid (HEDTA), and nitrilotriacetic acid (NTA).

In certain embodiments, the cosmetically acceptable excipient includes additional antioxidants such as a form of Vitamin E. Suitable forms of Vitamin E that may be included in the topical composition can be selected from alpha, beta, delta, and gamma tocopherols, and alpha, beta, delta and gamma tocotrienols, and combinations thereof.

In certain embodiments, the cosmetically acceptable excipient in the formulation includes a preservative. Suitable preservative agents include, for example, phenoxyethanol, a solution of paraben, pentanediol and sorbic acid, as well as silver complexes which are known under the commercial reference Surfacine® and other classes of substances set out in annex 6, parts A and B of the cosmetic regulations, i.e. a suitable preservative.

In certain embodiments, the cosmetically acceptable excipient includes a perfume oil. Suitable perfume oils include mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl-methylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include mainly the terpenes and balsams.

Essential oils of relatively low volatility, which are mostly used as aroma components, are also suitable as perfume oils, e.g. sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Other suitable oils include bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

In certain embodiments, the cosmetically acceptable excipient includes a perfume oil that is an essential oil selected from the group of a lavender oil, a bergamot oil, a eucalyptus oil, a chamomile oil, a melaleuca oil, or a combination thereof. In one embodiment, the cosmetically acceptable excipient includes a lavender oil, a chamomile oil, or a combination thereof.

In some embodiments, the formulations described herein may be formulated in any dermatological acceptable vehicle such as a serum, emulsion, cream, foam, spray, ointment, gel, lotion, or as a pad or roll-on applied formulation, which may contain ingredients to improve, modify, or stabilize the composition physically or cosmetically.

The formulations according to the disclosure may also contain one or one more additional cosmetically acceptable excipients as described below.

In some embodiments, the cosmetically acceptable excipient may be a Guerbet alcohol Guerbet alcohols based on fatty alcohols having from 6 to 18, preferably from 8 to 10 carbon atoms including cetyl alcohol, stearyl alcohol, cetearyl alcohol, oleyl alcohol, octyldodecanol, benzoate of C₁₂-C₁₅ alcohols, acetylated lanolin alcohol, etc.

The cosmetically acceptable excipient may further be an ester. For example, esters of linear C₆-C₂₄ fatty acids with linear C₃-C₂₄ alcohols, esters of branched C₆-C₁₃carboxyl acids with linear C₆-C₂₄ fatty alcohols, esters of linear C₆-C₂₄ fatty acids with branched alcohols, especially 2-ethylhexanol, esters of hydroxycarboxylic acids with linear or branched C₆-C₂₂ fatty alcohols, especially dioctyl malates, esters of linear and/or branched fatty acids with polyhydric alcohols (for example propylene glycol, dimer diol or trimer triol) and/or Guerbet alcohols, for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and mixtures thereof (obtained, for example, in the pressure removal of natural fats and oils, in the reduction of aldehydes from Roelen's oxosynthesis or in the dimerization of unsaturated fatty acids) with alcohols, for example, isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachidyl alcohol, gadoleyl alcohol, behenyl alcohol, erucyl alcohol and brassidyl alcohol and mixtures thereof (obtained, for example, in the high-pressure hydrogenation of methyl esters based on fats and oils or aldehydes from Roelen's oxosynthesis and as monomer fractions in the dimerization of unsaturated fatty alcohols).

Examples of such ester oils are isopropyl myristate, isopropyl palmitate, isopropyl stearate, isopropyl isostearate, isopropyl oleate, n-butyl stearate, n-hexyl laurate, n-decyl oleate, isooctyl stearate, iso-nonylstearate, isononyl isononanoate, 2-ethylhexylpalmitate, 2-hexyllaurate, 2-hexyldecylstearate, 2-octyldodecylpalmitate, oleyloleate, oleylerucate, erucyloleate, erucylerucate, cetearyl octanoate, cetyl palmitate, cetyl stearate, cetyl oleate, cetyl behenate, cetyl acetate, myristyl myristate, myristyl behenate, myristyl oleate, myristyl stearate, myristyl palmitate, myristyl lactate, propylene glycol dicaprylate/caprate, stearyl heptanoate, diisostearyl malate, octyl hydroxystearate, etc.

### Other Adjuvants

Diethylhexyl 2,6-naphthalate, di-n-butyl adipate, di(2-ethylhexyl)-adipate, di(2-ethyl hexyl)-succinate and diisotridecyl acelaat, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate. Esters of C₆-C₂₄ fatty alcohols and/or Guerbet alcohols with aromatic carboxylic acids, saturated and/or unsaturated, especially benzoic acid, esters of C₂-C₁₂ dicarboxylic acids with linear or branched alcohols having from 1 to 22 carbon atoms or polyols having from 2 to 10 carbon atoms and from 2 to 6 hydroxy groups.

### Natural or Synthetic Triglycerides Including Glyceryl Esters and Derivatives

Di- or triglycerides, based on C₆-C₁₈ fatty acids, modified by reaction with other alcohols (caprylic/capric triglyceride, wheat germ glycerides, etc.). Fatty acid esters of polyglycerin (polyglyceryl-n such as polyglyceryl-4 caprate, polyglyceryl-2 isostearate, etc. or castor oil, hydrogenated vegetable oil, sweet almond oil, wheat germ oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, borage oil, etc.

Waxes including esters of long-chain acids and alcohols as well as compounds having wax-like properties, e.g., carnauba wax, beeswax (white or yellow), lanolin wax, candelilla wax, ozokerite, japan wax, paraffin wax, microcrystalline wax, ceresin, cetearyl esters wax, synthetic beeswax, etc. Also, hydrophilic waxes as Cetearyl Alcohol or partial glycerides.

### Pearlescent Waxes:

Alkylene glycol esters, especially ethylene glycol distearate; fatty acid alkanolamides, especially coco fatty acid diethanolamide; partial glycerides, especially stearic acid monoglyceride; esters of polyvalent, unsubstituted or hydroxy-substituted carboxylic acids with fatty alcohols having from 6 to 22 carbon atoms, especially long-chained esters of tartaric acid; fatty substances, for example fatty alcohols, fatty ketones, fatty aldehydes, fatty ethers and fatty carbonates, which in total have at least 24 carbon atoms, especially lauryl and distearyl ether; fatty acids, such as stearic acid, hydroxystearic acid or behenic acid, ring-opening products of olefin epoxides having from 12 to 22 carbon atoms with fatty alcohols having from 12 to 22 carbon atoms and/or polyols having from 2 to 15 carbon atoms and from 2 to 10 hydroxy groups, and mixtures thereof.

### Hydrocarbon Oils:

Mineral oil (light or heavy), petrolatum (yellow or white), microcrystalline wax, paraffinic and isoparaffinic compounds, hydrogenated isoparaffinic molecules as polydecenes and polybutene, hydrogenated polyisobutene, squalane, isohexadecane, isododecane and others from plant and animal kingdom.

### Emulsifiers

Any conventionally usable emulsifier can be used for the compositions. Emulsifier systems may comprise for example: carboxylic acids and their salts: alkaline soap of sodium, potassium and ammonium, metallic soap of calcium or magnesium, organic basis soap such as Lauric, palmitic, stearic and oleic acid etc. Alkyl phosphates or phosphoric acid esters, acid phosphate, diethanolamine phosphate, potassium cetyl phosphate. Ethoxylated carboxylic acids or polyethylene glycol esters, PEG-n acylates. Linear fatty alcohols having from 8 to 22 carbon atoms, branched from 2 to 30 mol of ethylene oxide and/or from 0 to 5 mol propylene oxide with fatty acids having from 12 to 22 carbon atoms and with alkylphenols having from 8 to 15 carbon atoms in the alkyl group. Fatty alcohol polyglycol ether such as laureth-n, ceteareth-n, steareth-n, oleth-n. Fatty acid polyglycolether such as PEG-n stearate, PEG-n oleate, PEG-n cocoate. Monoglycerides and polyol esters. C12-C22 fatty acid mono- and di-esters of addition products of from 1 to 30 mol of ethylene oxide with polyols. Fatty acid and polyglycerol ester such as monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of those substance classes are also suitable. Fatty acid polyglycolesters such as monostearate diethylene glycol, fatty acid and polyethylene glycol esters, fatty acid and saccharose esters such as sucro esters, glycerol and saccharose esters such as sucro glycerides. Sorbitol and sorbitan, sorbitan mono- and di-esters of saturated and unsaturated fatty acids having from 6 to 22 carbon atoms and ethylene oxide addition products. Polysorbate-n series, sorbitan esters such as sesquiisostearate, sorbitan, PEG-(6)-isostearate sorbitan, PEG-(10)-sorbitan laurate, PEG-17-dioleate sorbitan. Glucose derivatives, C8-C22 alkyl-mono and oligo-glycosides and ethoxylated analogues with glucose being preferred as the sugar component. O/W emulsifiers such as methyl gluceth-20 sesquistearate, sorbitan stearate/sucrose cocoate, methyl glucose sesquistearate, cetearyl alcohol/cetearyl glucoside. W/O emulsifiers such as methyl glucose dioleate/methyl glucose isostearate. Sulfates and sulfonated derivatives, dialkylsulfosuccinates, dioctyl succinate, alkyl lauryl sulfonate, linear sulfonated paraffins, sulfonated tetrapropyene sulfonate, sodium lauryl sulfates, ammonium and ethanolamine lauryl sulfates, lauryl ether sulfates, sodium laureth sulfates, sulfosuccinates, acetyl isothionates, alkanolamide sulfates, taurines, methyl taurines, imidazole sulfates. Polysiloxane/polyalkyl/polyether copolymers and derivatives, dimethicone, copolyols, silicone polyethylene oxide copolymer, silicone glycol copolymer. Propoxylated or POE-n ethers (Meroxapols), Polaxamers or poly(oxyethylene)m-block-poly(oxypropylene)n-block(oxyethylene). Zwitterionic surfactants that carry at least one quaternary ammonium group and at least one carboxylate and/or sulfonate group in the molecule. Zwitterionic surfactants that are especially suitable are betaines, such as N-alkyl-N,N-dimethylammonium glycinates, cocoalkyldimethylammonium glycinate, N-acylaminopropyl-N,N-dimethylammonium glycinates, cocoacylaminopropyldimethylammonium glycinate and 2-alkyl-3-carboxymethyl-3-hydroxyethylimidazolines each having from 8 to 18 carbon atoms in the alkyl or acyl group and also cocoacylaminoethylhydroxyethylcarboxymethylglycinate, N-alkyl betaine, N-alkylaminobetaines. Alkylimidazolines, alkylopeptides, lipoaminoacides, self-emulsifying bases and the compounds as described in K. F. DePolo, A short textbook of cosmetology, Chapter 8, Table 8-7, p 250-251.

Nonionic bases such as PEG-6 beeswax (and) PEG-6 stearate (and) polyglyceryl-2-isostearate, glyceryl stearate (and) PEG-100 stearate, PEG-5 glyceryl stearate, sorbitan oleate (and) polyglyceryl-3 ricinoleate, sorbitan stearate and sucrose cocoate, glyceryl stearate and laureth-23, cetearyl alcohol and ceteth-20, cetearyl alcohol and polysorbate 60 and PEG-150 and stearate-20, cetearyl alcohol and cetearyl polyglucoside, cetearyl alcohol and ceteareth-20, cetearyl alcohol and PEG-40 castor oil, cetearyl alcohol and PEG-40 castor oil and sodium cetearyl sulfate, stearyl alcohol and steareth-7 and steareth-10, cetearyl alcohol and szeareth-7 and steareth-10, glyceryl stearate and PEG-75 stearate, propylene glycol ceteth-3 acetate, propylene glycol isoceth-3 acetate, cetearyl alcohol and ceteth-12 and oleth-12, PEG-6 stearate and PEG-32 stearate, PEG-6 stearate and ceteth-20 and steareth-20, PEG-6 stearate and ceteth-20 and glyceryl stearate and steareth-20, glyceryl stearate and ceteareth-20.

Anionic alkaline bases such as PEG-2 stearate SE, glyceryl stearate SE, propylene glycol stearate. Anionic acid bases such as cetearyl Alcohol and Sodium cetearyl sulfate, cetearyl alcohol and sodium lauryl sulfate, trilaneth-4 phosphate and glycol stearate and PEG-2 stearate, glyceryl stearate and sodium lauryl Sulfate. Cationic acid bases such as cetearyl alcohol and cetrimonium bromide.

### Surfactants

Examples of suitable mild surfactants, that is to say surfactants especially well tolerated by the skin, include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, .alpha.-olefin sulfonates, ethercarboxylic acids, alkyl oligoglucosides, fatty acid glucamides, alkylamidobetaines and/or protein fatty acid condensation products, the latter preferably being based on wheat proteins.

### Consistency Regulators/Thickeners and Rheology Modifiers

Silicium dioxide, magnesium silicates, aluminium silicates, polysaccharides or derivatives thereof for example hyaluronic acid, xanthan gum, guar-guar, agar-agar, alginates, carrageenan, gellan, pectines, or modified cellulose such as hydroxycellulose, hydroxypropylmethylcellulose. In addition polyacrylates or homopolymer of reticulated acrylic acids and polyacrylamides, carbomer (CARBOPOL types 980, 981, 1382, ETD 2001, ETD2020, ULTREZ 10) or SALCARE range such as SALCARE SC80 (steareth-10 allyl ether/acrylates copolymer), Salcare SC81 (acrylates copolymer), Salcare SC91 and Salcare AST (sodium acrylates copolymer/PPG-1 trideceth-6), SEPIGEL 305 (polyacrylamide/laureth-7), SIIVIULGEL NS and SIMULGEL EG (hydroxyethyl acrylate/sodium acryloyldimethyl taurate copolymer), STABILEN 30 (acrylates/vinyl isodecanoate crosspolymer), PEMULEN TR-1 (acrylates/C10-30 alkyl acrylate crosspolymer), LUVIGEL EM (sodium acrylates copolymer), ACULYN 28 (acrylates/beheneth-25 methacrylate copolymer), etc.

### Polymers

As anionic, zwitterionic, amphoteric and non-ionic polymers come into consideration, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyl-trimethylammonium chloride/acrylate copolymers, octyl acrylamide/methyl methacrylate-tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinyl caprolactam terpolymers and also optionally derivatized cellulose ethers and silicones. Furthermore, the polymer may be a crosspolymer. The crosspolymer may be poly(methylvinylether/maleic anhydric decadiene) crosspolymer.

In some embodiments, the polymer may also include a starch polymer.

### Biogenic Active Ingredients

Biogenic active ingredients are to be understood as meaning, for example, tocopherol, tocopherol acetate, tocopherol palmitate, deoxyribonucleic acid, retinol, bisabolol, allantoin, phytantriol, panthenol, AHA acids, amino acids, ceramides, pseudoceramides, essential oils, plant extracts and vitamin complexes.

### Antioxidants

Typical examples of such antioxidants are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotinoids, carotenes, lycopene and derivatives thereof, chlorogenic acid and derivatives thereof, lipoic acid and derivatives thereof (e.g. dihydrolipoic acid), aurothioglycose, propylthiouracil and other thiols (e.g. thioredoxin, glutathione, cysteine, cystine, cystamine and the glycosyl, N-acetyl, methyl, ethyl, propyl, amyl, butyl, lauryl, palmitoyl, oleyl, linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, hepta-thionine sulfoximine), also (metal) chelating agents (e.g. hydroxy fatty acids, palmitic acid phytic acid, lactoferrin), hydroxy acids (e.g. citric acid, lactic acid, malic acid), humic acid, bile acid, bile extracts, bilirubin, biliverdin, EDTA, EDDS, EGTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate) and also coniferyl benzoate of benzoin resin, rutinic acid and derivatives thereof, glycosylrutin, ferulic acid, furfurylidene glucitol, carnosine, butyl hydroxytoluene, butyl hydroxyanisole, nordihydroguaiaretic acid, trihydroxybutyrophenone, uric acid and derivatives thereof, mannose and derivatives thereof, superoxide dismutase, N-[3-(3,5-di-tert-butyl-4-hydroxyphenyl)propionyl]sulfanilic acid (and salts thereof, for example the disodium salts), selenium and derivatives thereof (e.g. selenium methionine), stilbene and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients. HALS (=“Hindered Amine Light Stabilizers”) compounds may also be mentioned.

### Preservatives and Bacteria-Inhibiting Agents

Suitable preservatives include, for example, Methyl-, Ethyl-, Propyl-, Butyl-parabens, Benzalkonium chloride, 2-Bromo-2-nitro-propane-1,3-diol, Dehydroacetic acid, Diazolidinyl Urea, 2-Dichloro-benzyl alcohol, DMDM hydantoin, Formaldehyde solution, Methyldibromoglutanitrile, Phenoxyethanol, Sodium Hydroxymethylglycinate, Imidazolidinyl Urea, Triclosan and further substance classes listed in the following reference: K. F. DePolo-A short textbook of cosmetology, Chapter 7, Table 7-2, 7-3, 7-4 and 7-5, p 210-219.

### Bacteria-Inhibiting Agents

Typical examples of bacteria-inhibiting agents are preservatives that have a specific action against gram-positive bacteria, such as 2,4,4′-trichloro-2′-hydroxydiphenyl ether, chlorhexidine (1,6-di(4-chlorophenyl-biguanido)hexane) or TCC (3,4,4′-trichlorocarbanilide). A large number of aromatic substances and ethereal oils also have antimicrobial properties. Typical examples are the active ingredients eugenol, menthol and thymol in clove oil, mint oil and thyme oil. A natural deodorizing agent of interest is the terpene alcohol farnesol (3,7,11-trimethyl-2,6,10-dodecatrien-1-ol), which is present in lime blossom oil. Glycerol monolaurate has also proved to be a bacteriostatic agent.

### Colorants

There may be used as colorants the substances that are suitable and permitted for cosmetic purposes, as compiled, for example, in the publication “Kosmetische Farbemittel” of the Farbstoffkommission der Deutschen Forschungsgemeinschaft, Verlag Chemie, Weinheim, 1984, pages 81 to 106.

In certain embodiments, the formulation may include a plant-based growth factor, a peptide, an antioxidant, a botanical agent, a polymer, a solvent, a chelating agent, a pH stabilizer, sodium hyaluronate, a surfactant, phenoxyethanol, or a combination thereof.

In some embodiments of the formulation, the plant-based growth factor may be included as a solution, where the plant-based growth factor may be included in the solution in about 1%, about 2%, about 3%, about 4%, or about 5% by weight, based on weight of the solution. The plant-based growth factor solution may be included in an amount of 1 wt % to about 10 wt %, about 2 wt % to about 9 wt %, about 3 wt % to about 8 wt %, or about 4 wt % to about 6 wt %, based on total weight of the formulation. In other embodiments, the plant-based growth factor may be included in an amount of about 1 wt %, about 2 wt %, about 3 wt %, about 4 wt %, about 5 wt %, about 6 wt %, about 7 wt %, about 8 wt %, about 9 wt %, or about 10 wt %, based on total weight of the formulation.

In some embodiments of the formulation, the peptide may be included in an amount of about 10 wt % to about 50 wt %, about 15 wt % to about 45 wt %, about 20 wt % to about 40 wt %, or about 25 wt % to about 35%, based on total weight of the formulation. In some embodiments, the peptide may be included in an amount of about 10 wt %, about 15 wt %, about 20 wt %, about 25 wt %, about 30 wt %, about 35 wt %, about 40 wt %, about 45 wt %, or about 50 wt %, based on total weight of the formulation.

In some embodiments, the antioxidant may be included in an amount of about 0.0001 wt % to about 1 wt %, about 0.001 wt % to about 0.9 wt %, about 0.05 w % to about 0.5 wt %, or about 0.01 wt % to about 0.05 wt %, based on total weight of the formulation.

In some embodiments, the botanical agent may be included in an amount of about 0.0001 wt % to about 1 wt %, about 0.001 wt % to about 0.5 wt %, about 0.01 wt % to about 0.1 wt %, or about 0.02 wt % to about 0.07 wt %, based on total weight of the formulation.

In some embodiments, each solvent individually or all solvents together may be present in the formulation at a concentration ranging from any of about 0.5 wt. %, about 1 wt. %, about 5 wt. %, about 10 wt. %, about 12 wt. %, about 15 wt. %, about 18 wt. %, about 20 wt. %, about 23 wt. %, or about 25 wt. % to any of about 28 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, or about 95 wt. %, based on total weight of the formulation. In certain embodiments, the concentration of all solvents, excluding water, in the formulation ranges from any of about 0.5 wt. %, about 1 wt. %, about 5 wt. %, about 10 wt. %, about 12 wt. %, about 15 wt. %, about 18 wt. %, about 20 wt. %, about 23 wt. %, or about 25 wt. % to any of about 28 wt. %, about 30 wt. %, about 35 wt. %, about 40 wt. %, about 45 wt. %, or about 50 wt. %, based on total weight of the topical formulation.

In some embodiments, the sodium hyaluronate may be included in an amount of about 0.001 wt % to about 5 wt %, about 0.01 wt % to about 4 wt %, about 0.1 wt % to about 3 wt %, or about 1 wt % to about 2 wt %, based on total weight of the formulation.

In some embodiments, the phenoxyethanol may be included in an amount of about 0.001 wt % to about 5 wt %, about 0.01 wt % to about 4 wt %, about 0.1 wt % to about 3 wt %, or about 1 wt % to about 2 wt %, based on total weight of the formulation.

In some embodiments, the pH stabilizer may be included in an amount of about 0.0001 wt % to about 1 wt %, about 0.001 wt % to about 0.9 wt %, about 0.01 wt % to about 0.75 wt %, or about 0.1 wt % to about 0.5 wt %, based on total weight of the formulation.

In another embodiment of the formulation, the formulation may include water, a PVM/MA decadiene crosspolymer, disodium EDTA, potassium hydroxide, acetyl hexapeptide-8, phenoxyethanol, potassium sorbate, acetic acid, glycerin, matricaria flower extract, sodium benzoate, citric acid, cucumber extract, octoxynol-9, sodium hyaluronate, ubiquinone, polysorbate 20, palmitoyl tripeptide-5, green tea extract, seaweed extract, polyvinylpyrrolidone (PVP), hydrolyzed vegetable protein, ethylhexylglycerin, biotin, butylene glycol, carbomer, palmitoyl pentapeptide-4, Nicotiana benthamiana hexapeptide-40 sh-oligopeptide-1, and Nicotiana benthamiana hexapeptide-40 sh-polypeptide-76.

In another embodiment of the formulation, the formulation may include water, a pvm/ma decadiene crosspolymer, tetrasodium glutamate diacetate, potassium hydroxide, polysorbate 20, pinanediol, camphanediol, butylene glycol, acetyl hexapeptide-8, phenoxyethanol, potassium sorbate, acetic acid, propanediol, polygutamic acid, palmitoyl tripeptide-5, panthenol, sodium hyaluronate, Dunaliella salina extract, tetradecyl aminobutyroylvalylaminobutyric urea trifluroacetate, glycerin, Macrocystis pyrifera (kelp) extract, PVP, hydrolyzed vegetable protein, ethylhexyl methacrylate/methyl methacrylate/VP copolymer, aminobutyric acid, oligopeptide-2, and dimethicone.

In another embodiment of the present disclosure, a method of treatment is provided. The method of treatment includes applying a formulation including a plant-based growth factor to the skin. The formulation may further include at least one of a peptide, an antioxidant, or a botanical agent. In another embodiment of the method of treatment, the method of treatment includes applying the formulation including a plant-based growth factor to the face. In yet another embodiment of the method of treatment, the method includes applying the formulation including a plant-based growth factor to an area under the eye.

In another embodiment, a method of treatment includes applying a first formulation including a plant-based growth factor and a first peptide mixture to skin and applying a second formulation including a plant-based growth factor and a second peptide mixture to the skin. The first peptide mixture includes acetyl hexapeptide-8, a hexapeptide-40, and a tripeptide, wherein the tripeptide is palmitoyl tripeptide-5. The second peptide mixture includes acetyl hexapeptie-8, a hexapeptide-40, and a tripeptide, wherein the tripeptide is palmitoyl tripeptide-5, palmitoyl pentapeptie-4, or a combination thereof.

In an embodiment of the method of treatments as described herein, the formulation may be applied daily for two weeks, wherein the appearance of fine lines and wrinkles is reduced at least 80%, at least 85%, at least 90%, at least 92%, or at least 95%. In some embodiments, the formulation may be applied daily for two weeks, wherein the skin appeared more youthful but at least 80%, at least 85%, at least 90%, at least 92%, or at least 95%. In some embodiments, the formulation may be applied daily for two weeks, wherein the skin appeared firm by at least 90%, at least 83%, at least 85%, at least 87%, at least 89%, at least 90%, at least 92%, or at least 95%.

In an embodiment of the method of treatments as described herein, the formulation may be applied daily for four weeks, wherein the appearance of fine lines and wrinkles is reduced at least 80%, at least 85%, at least 90%, at least 92%, at least 95%, at least 97%, at least 99%, or 100%. In some embodiments, the formulation may be applied daily for four weeks, wherein the skin appeared more youthful but at least 80%, at least 85%, at least 90%, at least 92%, at least 95%, at least 97%, at least 99%, or 100%. In some embodiments, the formulation may be applied daily for four weeks, wherein the skin appeared firm by at least 90%, at least 83%, at least 85%, at least 87%, at least 89%, at least 90%, at least 92%, at least 95%, at least 97%, at least 99%, or 100%.

In an embodiment of the method of treatments as described herein, the formulation may be applied daily for six weeks, wherein the appearance of fine lines and wrinkles is reduced at least 80%, at least 85%, at least 90%, at least 92%, at least 95%, at least 97%, at least 99%, or 100%. In some embodiments, the formulation may be applied daily for six weeks, wherein the skin appeared more youthful but at least 80%, at least 85%, at least 90%, at least 92%, at least 95%, at least 97%, at least 99%, or 100%. In some embodiments, the formulation may be applied daily for six weeks, wherein the skin appeared firm by at least 90%, at least 83%, at least 85%, at least 87%, at least 89%, at least 90%, at least 92%, at least 95%, at least 97%, at least 99%, or 100%. In some embodiments, the formulation may be applied daily for six weeks, wherein the skin radiance improved at least 90%, at least 83%, at least 85%, at least 87%, at least 89%, at least 90%, at least 92%, at least 95%, at least 97%, at least 99%, or 100%. In some embodiments, the formulation may be applied daily for six weeks, wherein there was a reduction in redness and reduction of pore size by at least 90%, at least 83%, at least 85%, at least 87%, at least 89%, at least 90%, at least 92%, at least 95%, at least 97%, at least 99%, or 100%.

According to the patient assessment questionnaire from the 6 week mark, the participants found that from the treatment, they experienced 100% reduced the appearance of fine lines and wrinkles, 100% skin looks more youthful and firmer, and 92% improved skin radiance. Participants also noted a reduction in redness and reduction of pore size. Similar results were also noted in the 8 week patient assessment questionnaire.

### ILLUSTRATIVE EXAMPLES

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1

A formulation in accordance with embodiments described herein was formulated into a serum having the composition described in Table 1 below. The formulation of Example 1 may be applied to an under the eye area.

TABLE 1
Serum Composition

| Ingredient | Amount (wt. %) |
|---|---|
| Water | 80 wt. %-90 wt. % |
| PVM/MA Decadiene Crosspolymer | 0.1 wt. %-1 wt. % |
| Tetrasodium Glutamate Diacetate | 0.01 wt. %-0.5 wt. % |
| Potassium Hydroxide | 0.1%-0.5 wt. % |
| Polysorbate 20 | 1 wt. %-5 wt. % |
| Pinanediol | Above 0 wt. %-0.5 wt. % |
| Camphanediol | Above 0 wt. %-0.5 wt. % |
| Butylene Glycol | 0.01 wt % to about 5 wt % |
| Acetyl Hexapeptide-8 | Above 0 wt. %-0.1 wt. % |
| Phenoxyethanol | 0.1 wt. %-1 wt. % |
| Potassium sorbate | Above 0 wt. %-0.5 wt. % |
| Acetic Acid | Above 0 wt. %-0.5 wt. % |
| Propanediol | 0.01 wt. %-0.3 wt. % |
| Polygutamic Acid | Above 0 wt. %-0.5 wt. % |
| Palmitoyl Tripeptide-5 | 0.01 wt % to about 5 wt % |
| Panthenol | 0.01 wt % to about 5 wt % |
| Sodium Hyaluronate | Above 0 wt. %-0.5 wt. % |
| Dunaliella Salina Extract | Above 0 wt. %-0.5 wt. % |
| Tetradecyl Aminobutyroylvalyl- | 1 wt. %-5 wt. % |
| aminobutyric Urea Trifluoroacetate | |
| Glycerin | Above 0 wt. %-1 wt. % |
| Macrocystis Pyrifera (Kelp) Extract | Above 0 wt. %-1 wt. % |
| PVP | Above 0 wt. %-1 wt. % |
| Hydrolyzed Vegetable protein | Above 0 wt. %-1 wt. % |
| Ethylhexyl Methacrylate/Methyl | Above 0 wt. %-0.5 wt. % |
| Methacrylate/VP Crosspolymer | |
| Aminobutyric Acid | Above 0 wt. %-1 wt. % |
| Oligopeptide-2 | 0.5 wt. %-5 wt. % |
| Dimethicone | 0.5 wt. %-5 wt. % |

### Example 2

A formulation in accordance with embodiments described herein was formulated into a serum having the composition described in Table 2 below. The formulation of Example 2 may be applied to the face.

TABLE 2
Serum Composition

| Ingredient | Amount (wt. %) |
|---|---|
| Water | 85 wt. %-95 wt. % |
| PVM/MA Decadiene Crosspolymer | 0.1 wt. %-1 wt. % |
| Disodium EDTA | 0.01 wt. %-1 wt. % |
| Potassium Hydroxide | 0.1%-1 wt. % |
| Acetyl Hexapeptide-8 | Above 0 wt. %-0.1 wt. % |
| Phenoxyethanol | 0.1 wt. %-5 wt. % |
| Potassium sorbate | Above 0 wt. %-0.5 wt. % |
| Polysorbate 20 | 0.01 wt. %-5 wt. % |
| Acetic Acid | Above 0 wt. %-0.5 wt. % |
| Glycerin | Above 0 wt. %-10 wt. % |
| Chamomilla Recutita (Matricaria) Flower | Above 0 wt. %-0.5 wt. % |
| Extract | |
| Sodium Benzoate | Above 0 wt. %-0.5 wt. % |
| Citric Acid | Above 0 wt. %-0.5 wt. % |
| Cucumis Sativus (Cucumber) Fruit Extract | Above 0 wt. %-0.5 wt. % |
| Octoxynol-9 | Above 0 wt. %-0.5 wt. % |
| Sodium Hyaluronate | Above 0 wt. %-0.5 wt. % |
| Ubiquinone | 0.01 wt % to about 5 wt % |
| Palmitoyl Tripeptide-5 | 0.001 wt % to about 5 wt % |
| Camellia Sinensis Leaf Extract | Above 0 wt. %-1 wt. % |
| Macrocystis Pyrifera (Kelp) Extract | Above 0 wt. %-1 wt. % |
| PVP | Above 0 wt. %-1 wt. % |
| Hydrolyzed Vegetable protein | Above 0 wt. %-1 wt. % |
| Ethylhexylglycerin | Above 0 wt. %-1 wt. % |
| Biotin | Above 0 wt. %-1 wt. % |
| Butylene Glycol | 1 wt. %-10 wt. % |
| Carbomer | 0.001 wt. %-0.75 wt. % |
| Palmitoyl Pentapeptide-4 | Above 0 wt. %-0.5 wt. % |
| Nicotiana Benthamiana Hexapeptide-40-sh- | Above 0 wt. %-0.5 wt. % |
| Oligopeptide-1 | |
| Nicotiana Benthamiana Hexapeptide-40-sh- | Above 0 wt. %-0.5 wt. % |
| Oligopeptide-76 | |

### Example 3

A formulation in accordance with embodiments described herein was formulated into a serum having the composition described in Table 3 below. The formulation of Example 3 may be applied to the face.

TABLE 3
Serum Composition

| Ingredient | Amount (wt. %) |
|---|---|
| Water | 60 wt. %-95 wt. % |
| PVM/MA Decadiene Crosspolymer | 0.1 wt. %-1 wt. % |
| Disodium EDTA | 0.01 wt. %-1 wt. % |
| Potassium Hydroxide | 0.1%-1 wt. % |
| Arigiraline (Acetyl Hexapeptide-8) | 15 wt. %-20 wt. % |
| Phenoxyethanol | 0.1 wt. %-5 wt. % |
| Chamomile Extract N10620WS | Above 0 wt. %-1 wt. % |
| Cucumber Extract N12113WS | Above 0 wt. %-1 wt. % |
| Sodium Hyaluronate 1% | Above 0 wt. %-5 wt. % |
| Co-Enzyme Q-10 | Above 0 wt. %-2 wt. % |
| Protasorb L-20 (Polysorbate 20 | 0.01 wt. %-5 wt. % |
| Syn-Coll (Glycerin) | Above 0 wt. %-10 wt. % |
| Green Tea P.E. 90 (Camellia Sinensis Leaf | Above 0 wt. %-5 wt. % |
| Extract) | |
| Skin Tightener ST | Above 0 wt. %-5 wt. % |
| Matrixyl MBAL (Palmitoyl Pentapeptide-4) | 1 wt. %-15 wt. % |
| Epitensive (Nicotiana Benthamiana | 0.5 wt. %-10 wt. % |
| Hexapeptide-40-sh-Oligopeptide-1) | |
| Reneseed (Nicotiana Benthamiana | 0.5 wt. %-10 wt. % |
| Hexapeptide-40-sh-Oligopeptide-76) | |

### Example 4—Consumer Test Results

The formulation according to Examples 2 and 3 were used in a six week consumer study. A consumer applied the formulation according to Example 2, blended at the time of application with the formulation of Example 3 twice daily. Baseline pictures and measurements were taken on the first day, and at two, four and six weeks. of the six-week consumer study.

After following the treatment plan for two weeks, the results of the measurements are presented in Table 4, indicating the speed at which positive results were obtained.

TABLE 4
Two-week Results of the Six-Week Study
Consumer Tested Results after Two Weeks

92% Reduced the appearance of fine lines and wrinkles
91% skin looked more youthful
83% Skin appeared firmer
84% Rejuvenated the skin
84% Reduced depth of wrinkles
84% Improved skin elasticity/bounce

### Example 5—Clinical Study Results

A clinical study was performed to investigate the efficacy, tolerance, and usability of a dual concentrated plant-based growth factor therapy including plant-based growth factors, peptides, and antioxidants designed to decrease the visible signs of aging, including fine lines, and wrinkles, texture, pore size, elasticity, skin color/clarity, redness, hydration and/or overall skin quality.

The study was conducted to assess the efficacy and tolerance of the daily application of a multi-correction treatment duo applied twice per day for 12 weeks. The participants in the study include females and/or males, ages 25-65.

The Efficacy Endpoints/Safety/Tolerability Endpoints on a Physician Assessment Scale were as follows:
Clinical Grading of Efficacy Parameters at baseline and weeks 2, 4, 8 and 12
Before and After photos required 2, 4, 8 and 12 weeks
Subject-completed self-assessment questionnaires at weeks 2, 4, 8 and 12
Clinical grading of objective and subjective irritation. Parameters at baseline and weeks 2, 4, 8, 10 and 12
Monitoring of adverse events (AEs) throughout the course of the study

The study included 30 participants ages 25-60, who applied a topical formulation including at least one plant-based growth factor in combination with a green tea antioxidant soothing cleanser, retinol eye wrinkle repair cream and a repair sunscreen.

According to the patient assessment questionnaire from the 2 week mark, 92% had reduced the appearance of fine lines and wrinkles, 91% found that the skin looked more youthful and 83% had skin that appeared firmer. After four weeks, 100% of participants said that the lines and wrinkles are visible reduced, skin looked more youthful and skin appeared firmer.

According to the patient assessment questionnaire from the 6 week mark, the participants found that from the treatment, they experienced 100% reduced the appearance of fine lines and wrinkles, 100% skin looks more youthful and firmer, and 92% improved skin radiance. Participants also noted a reduction in redness and reduction of pore size. Similar results were also noted in the 8 week patient assessment questionnaire.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

Reference throughout this specification to numerical ranges should not be construed as limiting and should be understood as encompassing the outer limits of the range as well as each number and/or narrower range within the enumerated numerical range.

The term “about”, when referring to a physical quantity, is to be understood to include measurement errors within, and inclusive of 10%. For example, “about 100° C.” should be understood to mean “100±10° C.”.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A formulation comprising:
at least one plant-based growth factor; and
at least one of a peptide; an antioxidant; or a botanical agent.

2. The formulation of claim 1, wherein the plant-based growth factor comprises plant-based synthetic human epidermal growth factor (EGF), a plant-based synthetic vascular endothelial growth factor (VEGF), a plant-based synthetic transforming growth factor (TGF), a plant-based synthetic insulin growth factor (IGF), or a combination thereof.

3. The formulation of claim 1, wherein the peptide comprises a hexapeptide, a tripeptide, a pentapeptide, or a combination thereof.

4. The formulation of claim 3, wherein the hexapeptide is selected from the group consisting of acetyl hexapeptide-8 and a hexapeptide-40.

5. The formulation of claim 4, wherein the hexapeptide-40 is Nicotiana benthamiana Hexapaeptide-40 sh-Oligopeptide-1 or Nicotiana benthamiana Hexapeptide-40 sh-Polypeptide-76.

6. (canceled)

7. (canceled)

8. The formulation of claim 1, wherein the peptide is acetyl hexapeptide-8, a hexapeptide-40, palmitoyl tripeptide-5, palmitoyl pentapeptide-4, or a combination thereof.

9. The formulation of claim 1, wherein the antioxidant is ubiquinone.

10. The formulation of claim 1, wherein the botanical agent comprises at least one of a chamomile extract, a cucumber extract, a green tea extract, a seaweed extract, or a combination thereof.

11. The formulation of claim 1, further comprising an alcohol.

12. (canceled)

13. The formulation of claim 1, further comprising a humectant.

14. (canceled)

15. (canceled)

16. (canceled)

17. The formulation of claim 1, further comprising a chelating agent.

18. (canceled)

19. The formulation of claim 1, further comprising a pH stabilizer.

20. (canceled)

21. The formulation of claim 1, further comprising a polymer.

22. (canceled)

23. (canceled)

24. The formulation of claim 1, further comprising a preservative.

25.-30. (canceled)

31. The formulation of claim 1, wherein the peptide is included in an amount of about 10 wt % to about 50 wt %, based on total weight of the formulation.

32. The formulation of claim 1, wherein the plant-based growth factor is included in an amount of 1 wt % to about 10 wt %, based on total weight of the formulation.

33. The formulation of claim 1, wherein the antioxidant is included in an amount of about 0.0001 wt % to about 1 wt %, based on total weight of the formulation.

34. The formulation of claim 1, wherein the botanical agent is included in an amount of about 0.0001 wt % to about 1 wt %, based on total weight of the formulation.

35. A method of treatment comprising applying a formulation comprising a plant-based growth factor to the skin.

36.-40. (canceled)

41. A method of treatment comprising applying a first formulation comprising a plant-based growth factor and a first peptide mixture to skin; and applying a second formulation comprising a plant-based growth factor and a second peptide mixture to the skin.

42.-44. (canceled)
